(19) United States
(12) Patent Application Publication
(10) Pub. No.: US 2018/0291355 A1
(43) Pub. Date: Oct. 11, 2018

(54) NATURAL RUBBER POLYMERASE GENE AND USES THEREOF

(71) Applicant: KOREA RESEARCH INSTITUTE OF BIOSCIENCE AND BIOTECHNOLOGY, Daejeon (KR)

(72) Inventor: Beung Tae RYU, Daejeon (KR)

(21) Appl. No.: 15/572,323

(22) PCT Filed: May 9, 2016
(86) PCT No.: PCT/KR2016/004827

§ 371 Date: Nov. 7, 2017

(30) Foreign Application Priority Data
May 7, 2015 (KR) 10-2015-0063748

(51) Int. Cl. C12N 9/10; C12N 15/82; C12P 5/02

(52) U.S. Cl. CPC C12N 9/1085; C12Y 205/0102; C12P 5/026; C12N 15/8243

(57) ABSTRACT

A rubber polymerase protein consisting of the amino acid sequence of SEQ ID NO: 2 for biosynthesis of natural rubber originating from a para rubber tree (Hevea brasiliensis). A gene encoding the rubber polymerase protein can be advantageously used for a technique of increasing a production amount of natural rubber from a rubber tree, a technique of producing natural rubber in other plants, microalgae, or microorganisms, or a technique for in vitro production of biorubber.

### TECHNICAL FIELD

The present invention relates to a natural rubber polymerase gene and uses thereof.

### BACKGROUND ART

At least ninety percent of natural rubber is produced from Southeast Asian countries like Malaysia, Indonesia, Thailand, and Myanmar. Although rubber is produced from about 2,000 kinds of plants, a para rubber tree is used as a main source of natural rubber since high quality rubber is found in an abundant amount and can be easily collected from a para rubber tree. In South Korea, at least 200,000 tons of natural rubber are consumed every year. Because the entire amount of the natural rubber is imported from foreign countries while demand for natural rubber increases continuously and an area of rubber plantation tends to decrease, a study on biosynthesis of natural rubber for replacing the natural rubber or development of other rubber source is required.

The para rubber tree (Hevea brasiliensis) simply known as a rubber tree belongs to Euphorbiaceae and is the economically most important tree of genus Hevea. A large amount of latex as a main raw material of natural rubber (i.e., cis-1,4-polyisoprene) can be produced from a para rubber tree, and at present moment, it is known as an almost single source of natural rubber that can be industrially used.

Biosynthesis of natural rubber in rubber tree occurs on a surface of a rubber particle which floats in latex, a cytoplasm of laticifer of a rubber tree. The first step of the rubber biosynthesis is a reaction for isomerization of IPP (isopentenyl diphosphate) to DMAPP (dimethylallyl pyrophosphate) catalyzed by an IPP isomerase, and rubber polymer is produced based on a continuous head-to-tail condensation of a 5-carbon intermediates catalyzed by a rubber transferase (or polymerase).

In the United States, Europe, Russia, or the like, attention is paid in recent years to dandelion as an alternative crop for producing natural rubber since it has high content of natural rubber and high rubber production amount per unit area. According to one natural rubber study group of the United States, it is reported that 10 to 20% of natural rubber component has been extracted from roots of Russian dandelion. In addition, compared to tree species which have to be grown for several years, dandelion has a short growth period. Since the production of natural rubber currently depends only on one rubber tree of genus Hevea, there is a risk of having reduced or interrupted production of natural rubber as possibly caused by diseases. As such, development of an alternative natural rubber crop is urgently needed to overcome those problems.

In this regard, the inventors of the present invention isolated from a para rubber tree (Hevea brasiliensis) a rubber polymerase for biosynthesis of natural rubber, and it is intended to achieve production of natural rubber in large quantities by using a gene encoding the natural rubber polymerase, or to use industrially the gene for producing natural rubber in large quantities in other plants, microalgae, or microorganisms. Furthermore, it is intended to use the gene for producing a large amount of biorubber by adding the rubber polymerase to a solution containing precursor materials, cofactors, and natural or artificial rubber particles that are required for in vitro rubber biosynthesis.

Meanwhile, in Korean Patent Registration No. 1281068, “Laticiferous tissue-specific SRPP promoter from Hevea brasiliensis and uses thereof” is described, and in Korean Patent Registration No. 0302100, “Recombinant microorganism expressing small rubber particle-associated protein (SRPP)” is described. However, the natural rubber polymerase gene of the present invention and uses thereof are not described therein.

### DETAILED DESCRIPTION OF THE INVENTION

### Technical Problems to be Solved

The present invention is devised under the circumstances described above. Specifically, the inventors of the present invention isolated a rubber polymerase gene for biosynthesis of natural rubber from a para rubber tree, and also found that, compared to a non-transgenic plant, a transgenic plant having overexpressed rubber polymerase by transformation of a Russian dandelion plant with a recombinant vector containing the gene has increased biosynthesis of natural rubber in plant like increased rubber content.

Furthermore, an antibody against the rubber polymerase which has been isolated in the present invention is prepared, and, by using the antibody, the rubber polymerase is obtained from a rubber tree latex based on immunoprecipitation. It is confirmed that, by adding an isolated rubber polymerase protein to a solution in which small-sized isoprenoid compound substrates as a precursor of natural rubber synthesis are mixed with rubber particles from which proteins are removed by using Triton-X 100 detergent, followed by an in vitro reaction for polymerization of isoprenoid monomers, production of large-sized isoprenoid polymer natural rubber can be achieved, and the present invention is completed accordingly.

### Technical Means for Solving the Problems

To solve the above problem, the present invention provides a rubber polymerase protein consisting of the amino acid sequence of SEQ ID NO: 2 for biosynthesis of natural rubber originating from a para rubber tree (Hevea brasiliensis).

The present invention further provides a gene encoding the aforementioned rubber polymerase protein.

The present invention further provides a recombinant vector containing the aforementioned gene.

The present invention further provides a host cell transformed with the aforementioned recombinant vector.

The present invention further provides a method for producing a transgenic plant with increased natural rubber content or increased molecular weight of a rubber polymer compared to a non-transgenic plant based on transformation of a plant cell with the aforementioned recombinant vector.

The present invention further provides a transgenic plant produced by the aforementioned method, and a seed of the transgenic plant.

The present invention further provides a composition for increasing natural rubber content in plant or increasing molecular weight of a rubber polymer containing the aforementioned gene as an effective ingredient.

The present invention further provides a method for increasing natural rubber content or increasing molecular weight of a rubber polymer in microorganism comprising transforming a microorganism cell with the aforementioned recombinant vector to express a gene encoding a rubber polymerase protein.

The present invention further provides a method for increasing natural rubber content in plant or increasing molecular weight of a rubber polymer comprising over-expressing a gene encoding a rubber polymerase protein based on transformation of a plant cell with a recombinant vector containing the aforementioned gene.

The present invention further provides a method for biosynthesis of biorubber by producing, either in a cell or in vitro, a recombinant rubber polymerase using a recombinant vector containing the aforementioned gene, or by isolating a rubber polymerase from a rubber tree plant followed by addition of substrates, cofactors, and rubber particles for in vitro biosynthesis of biorubber.

### Advantageous Effect of the Invention

The natural rubber biosynthesis polymerase gene of the present invention, which originates from a rubber tree plant, is a functional gene that can bring an improvement of natural rubber in plants in terms of quality and quantity, and it can contribute to enhancement of the productivity of natural rubber. Furthermore, the gene of the natural rubber biosynthesis polymerase of the present invention can be used for a technique for producing a large amount of natural rubber in other plants, microalgae, or microorganisms, and once an industrial crop for producing rubber as a useful resource material is developed, not only the natural rubber import dependency can be lowered but also the Korean national agenda of low carbon, green growth can be achieved by lowering the consumption of petroleum as a raw material of synthetic rubber.

### BRIEF DESCRIPTION OF THE DRAWINGS

FIG. 1 shows a result of Western blotting that is performed for rubber polymerase protein (named HvPep16) adhered to rubber particles of rubber tree latex, in which the Western blotting is performed after immunoprecipitation using an antibody against HvPep16 and SDS-PAGE.

FIG. 2 shows a result of measuring the rubber polymerase activity for producing an isoprenoid polymer as a rubber component after adding, to an in vitro reaction solution, the rubber polymerase which has been isolated by immunoprecipitation from rubber particles of rubber tree latex as shown in FIG. 1, in which the activity is expressed in terms of % compared to the activity of control group (i.e., IP carried out without adding any rubber particle protein). It was confirmed that, in accordance with an increase in the addition amount, the amount of produced rubber polymer has increased. It was also confirmed that the enzyme activity is lost when the enzyme is added after thermal inactivation.

FIG. 3 shows a binary vector which is constructed to introduce the rubber polymerase gene (named HvPep16) isolated from a rubber tree to Russian dandelion.

FIG. 4 shows a result of inducing forming of a transformant by adding the binary vector described in FIG. 3 to Agrobacterium (LBA4404) and inoculating the resulting bacteria to leaf tissue callus of Russian dandelion. The inoculated callus tissue was transferred to a regeneration induction medium containing hygromycin so that new shoots can grow only from transformed callus. It was shown in FIG. 4 that most of the callus tissues and regenerated shoots have died because of hygromycin, and only a very limited number of shoots, which are believed to be transformed, kept growing.

For FIG. 5, once roots are formed from the shoots survived in the hygromycin selection medium, they were transferred to a soil pot and allowed to grow for 6 weeks. Then, DNA was extracted from a small amount of leaf fragments, and determination was made by PCR using primers for the introduced rubber polymerase gene (HvPep16) to see whether or not the transformation has occurred. Total sixteen independent HvPep16-transgenic lines were obtained.

FIG. 6 shows a result of measuring the enzyme activity of a rubber polymerase in latex tissue, in which the measurement was made after collecting latex from roots of Russian dandelion transformant introduced with rubber polymerase gene (HvPep16). As a control group, a transformant which has been introduced with GUS gene was used. Latex was collected in an amount of 5 μl for each, and after adding it to a reaction buffer containing C¹⁴-IPP (isoprenoid monomer), the reaction was allowed to occur for 3 days. The activity of the rubber polymerase was determined by measuring the radioactivity (dpm, disintegrations per minute) as the amount of the C^{H}-labeled isoprenoid monomer transformed into an isoprenoid polymer (i.e., biosynthesis rubber).

FIG. 7 shows a result of measuring the rubber content in roots of the Russian dandelion transformant which has been introduced with rubber polymerase gene (HvPep16). As a control group, a transformant introduced with GUS gene was used.

### BEST MODE(S) FOR CARRYING OUT THE INVENTION

To achieve the purpose of the present invention, the present invention provides a rubber polymerase protein consisting of the amino acid sequence of SEQ ID NO: 2 for biosynthesis of natural rubber originating from a para rubber tree (Hevea brasiliensis).

The scope of the rubber polymerase protein according to the present invention includes a protein having an amino acid sequence represented by SEQ ID NO: 2, and also functional equivalents of the protein. The term “functional equivalent” indicates a protein having, as a result of addition, substitution, or deletion of an amino acid, at least 70%, preferably at least 80%, more preferably at least 90%, and even more preferably at least 95% sequence homology with the amino acid sequence represented by SEQ ID NO: 2, and it indicates a protein exhibiting substantially the same physiological activity as the protein represented by SEQ ID NO: 2. The expression “substantially the same physiological activity” means an activity of natural rubber biosynthesis.

The present invention further provides a gene encoding the aforementioned rubber polymerase protein. The gene encoding the rubber polymerase protein of the present invention may include the nucleotide sequence of SEQ ID NO: 1. Further, homologues of the nucleotide sequence are also within the scope of the present invention. Specifically, the above described gene may comprise a nucleotide sequence which has preferably at least 70%, more preferably at least 80%, still more preferably at least 90%, and most preferably at least 95% homology with the nucleotide sequence of SEQ ID NO: 1. The “sequence homology %” for a certain polynucleotide is identified by comparing a comparative region with two sequences that are optimally aligned. In this regard, a part of the polynucleotide in comparative region may comprise an addition or a deletion (i.e., a gap) compared to a reference sequence (without any addition or deletion) relative to the optimized alignment of the two sequences.

The present invention further provides a recombinant vector containing the aforementioned rubber polymerase gene for biosynthesis of natural rubber.

The term “recombinant” indicates a cell which replicates a heterogeneous nucleotide or expresses said nucleotide, or a peptide, a heterogeneous peptide, or a protein encoded by a heterogeneous nucleotide. Recombinant cell can express a gene or a gene fragment in the form of a sense or antisense, which are not found in natural state of cell. In addition, a recombinant cell can express a gene that is found in natural state, provided that said gene is modified and re-introduced into the cell by an artificial means.

The term “vector” is used herein to refer DNA fragment (s) and nucleotide molecules that are delivered to a cell. Vector can replicate DNA and be independently reproduced in a host cell. The terms “delivery system” and “vector” are often interchangeably used. The term “expression vector” means a recombinant DNA molecule comprising a desired coding sequence and other appropriate nucleotide sequences that are essential for the expression of the operatively-linked coding sequence in a specific host organism.

The vector of the present invention can be constructed as a vector which is typically used for cloning or expression. In addition, the vector of the present invention can be constructed by having a prokaryotic cell or an eukaryotic cell as a host. For example, when the vector of the present invention is an expression vector and a prokaryotic cell is employed as a host, a strong promoter for the initiation of transcription (e.g., pLλ promoter, trp promoter, lac promoter, T7 promoter, tac promoter and the like), and a ribosome binding site for the initiation of translation and a termination sequence for transcription/translation are generally comprised. When E. coli is employed as a host cell, a promoter and an operator region relating to the biosynthetic pathway of tryptophan in E. coli, and left-side promoter of phage λ (i.e., pLλ promoter) can be used as a regulation site.

Meanwhile, the vector which can be used for the present invention can be constructed by manipulating a plasmid (e.g., pSC101, ColE1, pBR322, pUC8/9, pHC79, pGEX series, pET series, pUC19 and the like), a phage (e.g., λgt4·λB, λ-Charon, λΔz1, M13 and the like) or a virus (e.g., SV40 and) that are often used in the pertinent art.

On the other hand, when the vector of the present invention is an expression vector and an eukaryotic cell is employed as a host, a promoter originating from genome of mammalian cell (e.g., metallothionein promoter) or a promoter originating from mammalian virus (e.g., adenovirus late promoter, vaccinia virus 7.5K promoter, SV40 promoter, cytomegalovirus promoter and tk promoter of HSV) can be utilized. As a termination sequence for transcription, a polyadenylation sequence is generally comprised.

The vector of the present invention may include as a selection marker an antibiotics-resistant gene that is conventionally used in the pertinent art, and the example includes a gene which is resistant to ampicillin, gentamycin, carbenicillin, chloramphenicol, streptomycin, kanamycin, geneticin, neomycin and tetrcycline.

The recombinant vector of the present invention is preferably a plant expression vector.

A preferred example of the plant expression vector is Ti-plasmid vector which can transfer a part of itself, i.e., so called T-region, to a plant cell when the vector is present in an appropriate host such as Agrobacterium tumefaciens. Other types of Ti-plasmid vector (see, EP 0 116 718 B1) are currently used for transferring a hybrid gene to protoplasts that can produce a new plant by appropriately inserting a plant cell or hybrid DNA to a genome of a plant. Especially preferred form of Ti-plasmid vector is a so called binary vector which has been disclosed in EP 0 120 516 B1 and U.S. Pat. No. 4,940,838. Other vector that can be used for introducing the DNA of the present invention to a host plant can be selected from a double-stranded plant virus (e.g., CaMV), a single-stranded plant virus, and a viral vector which can be originated from Gemini virus, etc., for example a non-complete plant viral vector. Use of said vector can be advantageous especially when a plant host cannot be appropriately transformed.

For the plant expression vector according to the present invention, a promoter can be any of SRPP (small rubber particle-associated protein), CaMV 35S, actin, ubiquitin, pEMU, MAS or histone promoter, but not limited thereto. The term “promoter” means a DNA molecule to which RNA polymerase binds in order to initiate its transcription, and it corresponds to a DNA region upstream of a structural gene. The term “plant promoter” indicates a promoter which can initiate transcription in a plant cell. The term “constitutive promoter” indicates a promoter which is active in most of environmental conditions and development states or cell differentiation states. Since a transformant can be selected with various mechanisms at various stages, a constitutive promoter can be preferable for the present invention. Therefore, a possibility for choosing a constitutive promoter is not limited herein.

For the plant expression vector of the present invention, any conventional terminator can be used. Examples thereof include, nopaline synthase (NOS), rice α-amylase RAmy1 A terminator, phaseoline terminator, and a terminator for octopine gene of Agrobacterium tumefaciens, etc., but are not limited thereto.

The present invention further provides a host cell transformed with the aforementioned recombinant vector. Any kind of a host cell including microalgae and microorganisms that are known in the pertinent art can be used if stable and continuous cloning and expression of the vector of the present invention can be achieved by using it. Examples include strains belonging to the genus Bascillus such as E. coli JM109, E. coli BL21, E. coli RR1, E. coli LE392, E. coli B, E. coli X 1776, E. coli W3110, Bascillus subtilus, Bascillus thuringiensis, and the like, Salmonella typhimurium, intestinal flora and strains such as Serratia marcescens and various Pseudomonas Spp. and the like.

In addition, when the vector of the present invention is transformed in an eukaryotic cell, a host cell such as Saccharomyce cerevisiae, an insect cell, a human cell (e.g., CHO cell line (Chinese hamster ovary), W138, BHK, COS-7, 293, HepG2, 3T3, RIN and MDCK cell line), a plant cell line and the like can be used. Preferably, the host cell is a plant cell.

When a host cell is a prokaryotic cell, delivery of the vector of the present invention into a host cell can be carried out according to CaCl₂ method, Hanahan's method (Hanahan, D., J. Mol. Biol., 166:557-580 (1983)), and an electroporation method, etc. In addition, when a host cell is an eukaryotic cell, the vector of the present invention can be delivered into a host cell according to a microscopic injection method, calcium phosphate precipitation method, an electroporation method, a liposome-mediated transformation, DEAE-dextran treatment method and a gene bombardment method, etc.

The present invention further provides a method for producing a transgenic plant with increased natural rubber content or increased molecular weight of a rubber polymer compared to a non-transgenic plant comprising:

transforming a plant cell with a recombinant vector containing the gene, and

regenerating a transgenic plant from the transformed plant cell.

In the present invention, the molecular weight of a rubber polymer can be weight average molecular weight, but it is not limited thereto.

Plant transformation means any method by which DNA is delivered to a plant. Such transformation method does not necessarily need a period for regeneration and (or) tissue culture. Transformation of plant species is now quite general not only for dicot plants but also for monocot plants. In principle, any transformation method can be used for introducing a hybrid DNA of the present invention to appropriate progenitor cells. The method can be appropriately selected from a calcium/polyethylene glycol method for protoplasts, an electroporation method for protoplasts, a microscopic injection method for plant components, a particle bombardment method for various plant components (DNA or RNA-coated), or a (non-complete) viral infection method in Agrobacterium tumefaciens mediated gene transfer by plant invasion or transformation of fully ripened pollen or microspore, etc. A method preferred in the present invention includes Agrobacterium mediated DNA transfer. In particular, so-called binary vector technique as disclosed in EPA 120 516 and U.S. Pat. No. 4,940,838 can be preferably used for the present invention.

The method described in the above comprises transforming a plant cell with the recombinant vector of the present invention and the transformation can be mediated by Agrobacterium tumefaciens. In addition, the method described in the above comprises regenerating a transgenic plant from the transformed plant cell. Any method that is known in the pertinent art as a method of regenerating a transgenic plant from a transformed plant cell can be used for the present invention.

The “plant cell” that can be used for the plant transformation in the present invention can be any type of plant cell. It includes a cultured cell, a cultured tissue, a cultured organ or a whole plant. It is preferably a cultured cell, a cultured tissue, or a cultured organ. It is more preferably a cultured cell in any form.

The term “plant tissue” can be either differentiated or undifferentiated plant tissue, including root, stem, leaf, pollen, seed, cancerous tissue and cells having various shape that are used for culture, i.e., single cell, protoplast, bud and callus tissue, but not limited thereto. Plant tissue can be in planta or in a state of organ culture, tissue culture or cell culture.

The present invention further provides a transgenic plant produced by the aforementioned method, and a seed of the transgenic plant.

According to one embodiment of the present invention, the plant can be any rubber plant capable of producing rubber, and it can be para rubber tree, Indian rubber tree, ceara rubber tree, Arabian rubber tree, thistle, lettuce, Russian dandelion, or guayule rubber, and preferably a plant like para rubber tree, Russian dandelion, or guayule, but it is not limited thereto.

The present invention further provides a method for increasing natural rubber content or increasing molecular weight of a rubber polymer in microorganism comprising transforming a microorganism cell with the aforementioned recombinant vector to express a gene encoding a rubber polymerase protein.

Other than the transformation technique using a recombinant vector described above, the method of the present invention can employ a non-GMO technique to increase the natural rubber content in a plant. Examples of the non-GMO technique include a method using gene scissors like zinc finger nuclease and TALEN (transcription activator-like effector nuclease), a method of inducing mutagenesis like oligonucleotide-directed mutagenesis, cisgenesis and intragenesis, and RNA directed DNA methylation, grafting, reverse-crossing, and agroinfiltration, but it is not limited thereto.

The present invention further provides a composition for increasing natural rubber content in plant or increasing molecular weight of a rubber polymer containing the aforementioned gene as an effective ingredient. The composition of the present invention contains, as an effective ingredient, a gene encoding the rubber polymerase protein consisting of the amino acid sequence of SEQ ID NO: 2 for biosynthesis of natural rubber originating from Hevea brasiliensis, and by transforming a plant cell with the gene or a recombinant vector containing the gene, the natural rubber content in plant or molecular weight of a rubber polymer can be increased.

The present invention further provides a method for increasing natural rubber content in plant or increasing molecular weight of a rubber polymer comprising over-expressing a gene encoding a rubber polymerase protein based on transformation of a plant cell with a recombinant vector containing the aforementioned gene.

The present invention still further provides a method for in vitro biosynthesis of biorubber comprising:

producing, either in a cell or in vitro, a recombinant rubber polymerase using a recombinant vector containing the aforementioned gene, or isolating and producing a rubber polymerase in a plant tissue, and

adding substrates, cofactors, and rubber particles to the produced rubber polymerase protein for reaction.

The method of the present invention is a method for in vitro biosynthesis of biorubber, in which the first step is a step for producing a recombinant rubber polymerase protein in a transformed microorganism by transforming a microorganism with the gene of the present invention. The method for transforming a microorganism with the gene of the present invention is the same as those described above. Examples of the usable microorganism include Escherichia coli, yeast, and Pseudomonas, but it is not limited thereto. Furthermore, the recombinant rubber polymerase protein can be obtained by an in vitro protein synthesis method. By extracting the rubber polymerase present in plant tissues followed by isolation and purification, the protein can be also obtained.

The second step is to add substrates, cofactors, and rubber particles to the produced recombinant rubber polymerase protein for having a reaction. Examples of the usable substrate include isopentenyl pyrophosphate, farnesyl pyrophosphate, and geranyl geranyl pyrophosphate, and examples of the cofactors include magnesium ion and/or manganese ion. The rubber particles can be either isolated from a plant or artificially prepared.

Hereinbelow, the present invention is explained in greater detail in view of the Examples. However, it is evident that the following Examples are given only for exemplification of the present invention and by no means the present invention is limited to the following Examples.

### EXAMPLES

### Example 1. Sequence Analysis of Protein and Gene of Rubber Polymerase Originating from Para Rubber Tree

After creating a scratch in laticifer tissues on a surface of para rubber tree (Hevea brasiliensis), latex emulsion flowing out of the scratch was collected and stored in ice. The latex was admixed with a phosphate buffer and subjected to ultra-high speed centrifugation to separate a rubber particle layer from an upper layer. The separated rubber particle layer was washed 3 times with the phosphate buffer, and the rubber particle proteins that are adhered on the rubber particles were separated by using 0.02% Triton-X. The separated rubber particle proteins were subjected to isolation and purification based on a series of chromatography or electrophoresis. The activity of rubber biosynthesis was then measured for the extracted protein, and by separating the proteins showing the activity, three peptide fragment sequences were obtained (internal peptide 1: LTEGFYSLR (SEQ ID NO: 3), internal peptide 2: RDFESGLDAAFAACR (SEQ ID NO: 4), and internal peptide 3: YALLDYSEPR (SEQ ID NO: 5)).

To obtain the whole sequence information of the corresponding protein by using the information of those three peptide sequences, total RNA was extracted from the latex solution, and a large-scale RNA sequencing was carried out. According to blasting of assembled database, a DNA contig corresponding to the separated fragment peptide sequence was obtained from the RNA sequencing database. Thereafter, by connecting the sequence according to blasting in order the raw database, the whole sequence of the gene was obtained (SEQ ID NO: 1), and the gene was named HvPep16. By translating the obtained gene sequence into an amino acid sequence, the whole sequence of the rubber polymerase protein was obtained (SEQ ID NO: 2).

### Example 2. In Vitro Rubber Production after Isolation and Extraction of Rubber Polymerase Originating from Rubber Tree

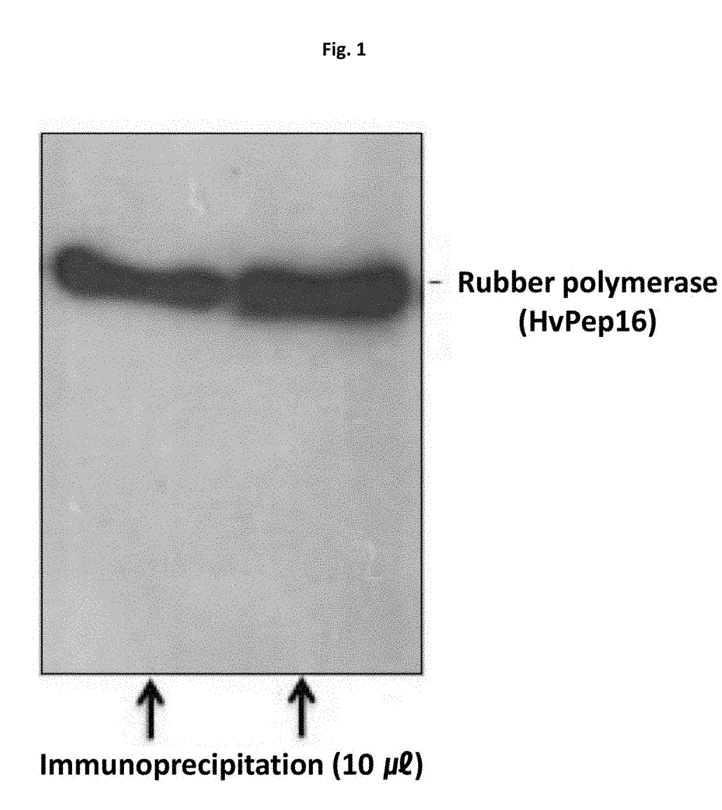

The rubber polymerase protein present in latex of a para rubber tree was obtained by immunoprecipitation using an antibody against the rubber polymerase which has been isolated in the present invention (FIG. 1). The antibody was produced by selecting, from the amino acid sequence of HvPep16 protein, 3 internal peptide sequences which have high possibility of inducing an antibody, and after conjugation to beads, the antibody was injected to a rabbit. After boosting for 3 times, blood was collected to obtain the antibody. By adding a solution containing isoprenoid compound substrates which are small-sized precursors for biosynthesis of natural rubber and rubber particles from which proteins are removed by Triton-X 100 detergent followed by having an in vitro reaction for 3 days, it was confirmed that the isoprenoid monomers can be polymerized to yield a large-sized biorubber isoprenoid polymer. As for the precursor substrate compound, FPP (farnesyl pyrophosphate) having 15 carbons was used. As for the isoprenoid monomer, ¹⁴C-radiolabeled IPP (isopentenyl pyrophosphate) having 5 carbons was used. After the in vitro reaction for 3 days at 37° C., the produced isoprenoid polymer was separated into a rubber component (with carbon atom number of 2,000 or higher) and a non-rubber component having small size. The separation was made by using the method described by Asawatreratanakul, et. al. (2003, Eur. J. Biochem. 270: 4671-4680).

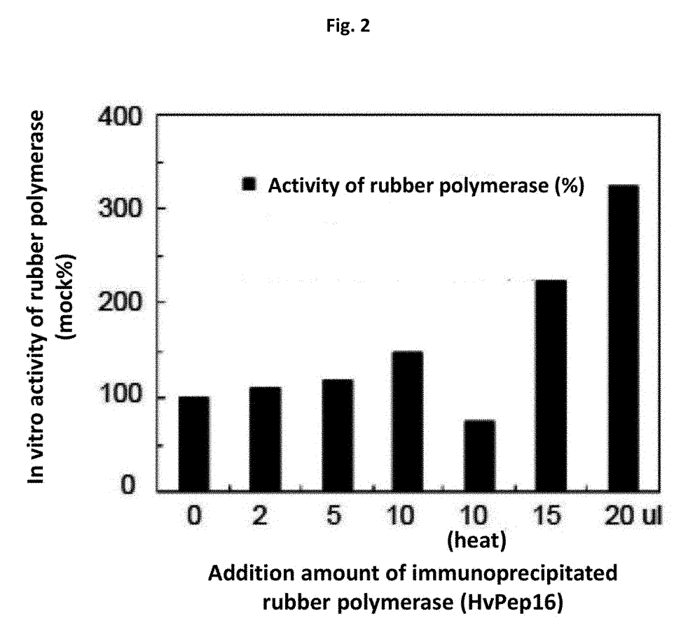

As a result, it was found that the sample added with the rubber polymerase originating from a rubber tree, which has been isolated in the present invention, yields 3 time or higher synthesis of isoprenoid polymer with rubber component (with carbon atom number of 2,000 or higher) compared to the control group (i.e., IP carried out without adding rubber particle proteins) (FIG. 2), and thus it was able to confirm the activity of a rubber polymerase originating from a rubber tree, which has been isolated in the present invention.

### Example 3. Production of Transformant by Introduction of Rubber Polymerase Gene Originating from Para Rubber Plant to Russian Dandelion and Characteristics Analysis of Transformant

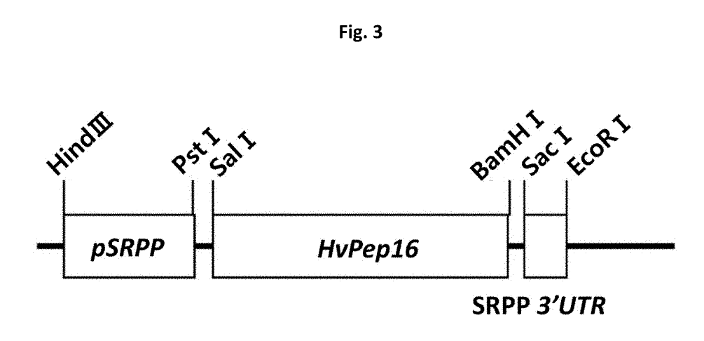

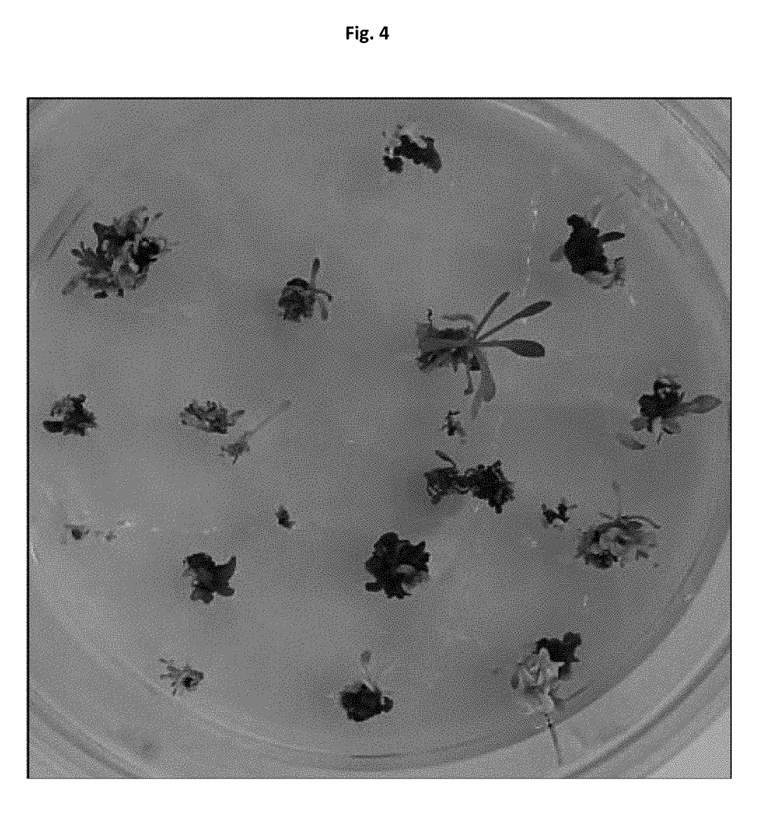

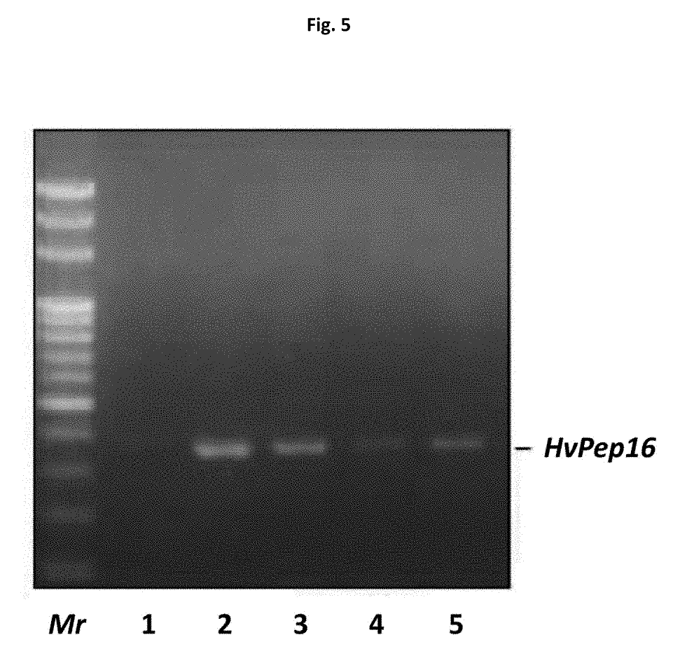

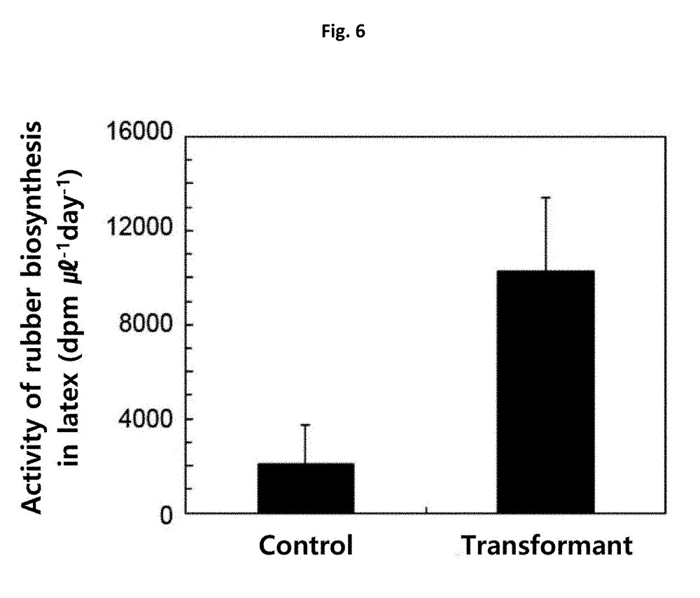

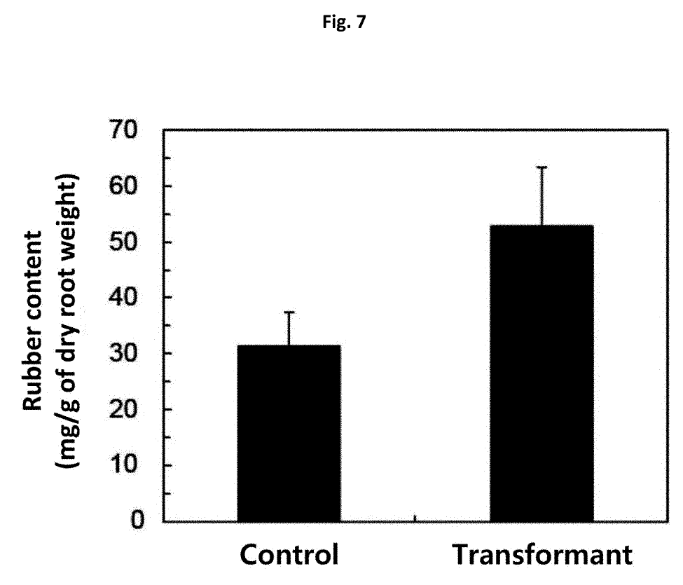

In order to determine the function of the gene and protein of a rubber polymerase isolated from a para rubber tree, the rubber polymerase gene originating from a para rubber tree (i.e., HvPep16) was introduced to Russian dandelion, which allows relatively easy transformation. The constitution of the produced vector for transformation is shown in FIG. 3. As for the promoter for gene expression, latex tissue-specific expression promoter pSRPP, which has been recently isolated by the study group of the present application, was used (see, U.S. Pat. No. 8,907,074). Transformation of Russian dandelion was carried out by using the method described by Bae, et. al. (2005, Plant Cell, Tissue and Organ Culture 80: 51-57). The transformed Russian dandelion survived in hygromycin-selection medium (see, FIG. 4) was transferred to a soil pot and allowed to grow for 6 weeks. Thereafter, genomic DNA was extracted from the leaf of the plant, and correct introduction of the foreign gene was determined by PCR using vector primers (FIG. 5). The Russian dandelion transformant which is finally determined to have the rubber polymerase of a rubber tree introduced therein was allowed to grow for 4 more weeks in an LED growth chamber. To enhance the expression of the introduced gene by activating the promoter pSRPP, the plant was treated at low temperature (5° C.) for 3 nights (8 hours), and then allowed to grow for 2 days at normal growth temperature (22° C. day/night). Thereafter, the activity of rubber biosynthesis and rubber content in root tissues were measured. As a control group, Russian dandelion introduced with GUS gene was used. The roots of Russian dandelion transformant introduced with rubber polymerase (HvPep16) showed the activity or rubber biosynthesis that is higher by 5 times compared to the control group (FIG. 6). This result (FIG. 6) indicates that the introduced rubber polymerase originating from a para rubber tree is expressed in latex tissues of Russian dandelion to exhibit the activity of rubber biosynthesis. The method used therefor is described in the above BRIEF DESCRIPTION OF THE DRAWINGS relating to FIG. 6, and unlike Example 2, the rubber particles washed with a detergent were not added to the reaction solution. Furthermore, the roots of the Russian dandelion transformant introduced with the rubber polymerase gene show the rubber content that is increased by 1.7 times compared to the control group (FIG. 7).

1. A rubber polymerase protein consisting of the amino acid sequence of SEQ ID NO: 2 for biosynthesis of natural rubber originating from a para rubber tree (Hevea brasiliensis).

2. A gene encoding the rubber polymerase protein of claim 1.

3. The gene encoding the rubber polymerase protein according to claim 2, wherein the gene consists of the nucleotide sequence of SEQ ID NO: 1.

4. A recombinant vector containing the gene of claim 2.

5. A host cell transformed with the recombinant vector of claim 4.

6. The host cell according to claim 5, wherein the host cell is either a plant or a microorganism.

7. A method for producing a transgenic plant with increased natural rubber content or increased molecular weight of a rubber polymer compared to a non-transgenic plant comprising:
transforming a plant cell with the recombinant vector of claim 4, and
regenerating a transgenic plant from the transformed plant cell.

8. A transgenic plant produced by the method of claim 7 having increased natural rubber content or increased molecular weight of a rubber polymer compared to a non-transgenic plant.

9. A transformed seed of the transgenic plant of claim 8.

10. A composition for increasing natural rubber content in plant or increasing molecular weight of a rubber polymer containing the gene of claim 2 as an effective ingredient.

11. A method for increasing natural rubber content or increasing molecular weight of a rubber polymer in microorganism comprising transforming a microorganism cell with the recombinant vector of claim 4 to express a gene encoding a rubber polymerase protein.

12. A method for in vitro biosynthesis of biorubber comprising:
producing, either in a cell or in vitro, a recombinant rubber polymerase using the recombinant vector of claim 4, or isolating and producing a rubber polymerase in a plant tissue, and
adding a substrate, a cofactor, and a rubber particle to the produced rubber polymerase protein for reaction.

13. The method of claim 12, wherein the recombinant rubber polymer is produced in the cell.

14. The method of claim 12, wherein the recombinant rubber polymer is produced in vitro.

15. The method of claim 12, wherein the rubber polymer in the plant tissue is isolated and produced.

16. The method of claim 12, wherein the substrate is selected from the group consisting of isopentenyl pyrophosphate, farnesyl pyrophosphate, geranyl geranyl pyrophosphate, and a combination thereof.

17. The method of claim 12, wherein the cofactor is selected from the group consisting of magnesium ion, manganese ion, and a combination thereof.

18. The method of claim 12, wherein the rubber particle is either isolated from a plant or artificially prepared.


SEQUENCE LISTING (5 sequences)

SEQ ID NO: 1 - DNA, 1581 bp, Hevea brasiliensis
atgtcatcgattcccccaaattcaccctggagaggatgcgcagcattctttccacaacctgtatctgaaagtaatcaatctgcagctcaatttcttactgaaactgtctactactttaattgccgtgtaataaatcctcctttagatgatatcgaatgcagaagcagtgatggcgaacaagacatccttcgccatgtcttccgttgggatactgcaccttatcaacacgtttttcagcatggattcgaagcaaggcgtcaaggaggtactcccgatgaaatttacttcaatttggatcactatgttcatcatagtggcagacctcttgattccactaggcctgccacgcatgtcttcattagcaccacccttagcagtgcttggtatccaactctccctgatgggacacaagaagtgtatcgttatgagatatatgcaccagggggtatttgggttgctgagacacttggagatcgttacagatatcctgcccaagatgaggttgcctttgttgctggcatagcccctcaatacattcgctctgctcaattgttcagacttacccatagtggaaggtatacaagacgggagagggtggataacagactaattgtcaaccgtaattacaatcctcaatcacatccctcaagagtgctccccattcagaggcctgtatttgattatattcttaatggtaggagggaactcttaaatcttgttatctacaatcctaattctgcagctggagcaaatgataacgcatcaagagagaaacgagaagtctcctatgacgtcattgattggtataccgacaaagtggcagatgttggcacttacataaatgctgcatttcagtcagctcgtaatcgagacgaagtctatttattcatgaaaaatgagtacgttcttgtaaactatgctccaggaacaaccaatgatagggtgattaatgggccacttcttatttgcgatgggtttccatcacttacagatacagcatttgcagagttcggagtagattgtgcttttagatctcatcatggaaatgaagcattcatcttttctgggaatctttgtgcacgtatagactatgcacctggcactctcaatgacaagatactcaggggtccaatgcccattggtaccatgttccccttcttcaagaggacagtatttgagacgagtattgatgcagcttttgaggcaaccgctaccaatgaggcttatctgttcaaagatgatcaatatgctcttatagactattcggaacccagaaggattgctatccgtaaaataactgaaggcttttatagtttgagaggtactattttcgaaagtggaattgatgcggcctttgcttcttccaggaaaaatgaggcgtaccttttcaagggagatcagtatgcactcattaattttgctccaggaacaaccaatgactacatcattggtggtgtgaagccaatcctccccagttggcctagtcttcgaaacatattacctcgcaaaaatcgtggccttgacattcatgaacactctcataatactcctgaacccgatagagacagggatgatctttga

SEQ ID NO: 2 - protein, 526 aa, Hevea brasiliensis
MSSIPPNSPWRGCAAFFPQPVSESNQSAAQFLTETVYYFNCRVINPPLDDIECRSSDGEQDILRHVFRWDTAPYQHVFQHGFEARRQGGTPDEIYFNLDHYVHHSGRPLDSTRPATHVFISTTLSSAWYPTLPDGTQEVYRYEIYAPGGIWVAETLGDRYRYPAQDEVAFVAGIAPQYIRSAQLFRLTHSGRYTRRERVDNRLIVNRNYNPQSHPSRVLPIQRPVFDYILNGRRELLNLVIYNPNSAAGANDNASREKREVSYDVIDWYTDKVADVGTYINAAFQSARNRDEVYLFMKNEYVLVNYAPGTTNDRVINGPLLICDGFPSLTDTAFAEFGVDCAFRSHHGNEAFIFSGNLCARIDYAPGTLNDKILRGPMPIGTMFPFFKRTVFETSIDAAFEATATNEAYLFKDDQYALIDYSEPRRIAIRKITEGFYSLRGTIFESGIDAAFASSRKNEAYLFKGDQYALINFAPGTTNDYIIGGVKPILPSWPSLRNILPRKNRGLDIHEHSHNTPEPDRDRDDL

SEQ ID NO: 3 - protein, 9 aa, Hevea brasiliensis
LTEGFYSLR

SEQ ID NO: 4 - protein, 15 aa, Hevea brasiliensis
RDFESGLDAAFAACR

SEQ ID NO: 5 - protein, 10 aa, Hevea brasiliensis
YALLDYSEPR